(19) United States
(12) Patent Application Publication
(10) Pub. No.: US 2023/0270378 A1
(43) Pub. Date: Aug. 31, 2023

(54) ELECTRONIC DEVICE FOR SLEEP MONITORING AND OPERATING METHOD THEREOF

(71) Applicant: Samsung Electronics Co., Ltd., Suwon-si (KR)

(72) Inventors: Minhee HWANG, Suwon-si (KR); Seongho CHO, Suwon-si (KR); Hyogil KIM, Suwon-si (KR); Jeongmin PARK, Suwon-si (KR)

(21) Appl. No.: 18/141,186

(22) Filed: Apr. 28, 2023

Related U.S. Application Data
(63) Continuation of application No. PCT/KR2022/015746, filed on Oct. 17, 2022.

(30) Foreign Application Priority Data
Oct. 26, 2021 (KR) 10-2021-0143568

(51) Int. Cl. A61B 5/00; G16H 40/67; G16H 10/60

(52) U.S. Cl. CPC A61B 5/4818; A61B 5/7275; A61B 5/7282; A61B 5/0004; G16H 40/67; G16H 10/60; A61B 5/681

(57) ABSTRACT

An electronic device for sleep monitoring and an operation method includes a communication circuit, at least one sensor, and at least one processor. The at least one processor is configured to obtain biometric information of a user during the user's sleep to determine a reference condition for detecting a sleep breathing disorder event based on underlying disease information of the user, and to detect a sleep breathing disorder event based on the biometric information and the reference condition. The processor provides a user interface for the sleep breathing disorder event as the sleep breathing disorder event is detected.

### CROSS-REFERENCE TO RELATED APPLICATION(S)

This application is a continuation application, claiming priority under § 365(c), of an International application No. PCT/KR2022/015746, filed on Oct. 17, 2022, which is based on and claims the benefit of a Korean patent application number 10-2021-0143568, filed on Oct. 26, 2021, in the Korean Intellectual Property Office, the disclosure of which is incorporated by reference herein in its entirety.

### TECHNICAL FIELD

Various embodiments disclosed in this document relate to an electronic device for sleep monitoring and an operation method thereof.

### BACKGROUND ART

With the recent development of mobile communication technology, the use of portable or mobile electronic devices (e.g., smartphones and wearable devices) has become common, and various functions are provided through such electronic devices. For example, a healthcare service of consistently monitoring a user's biometric data or sleep state and managing health through an electronic device may be provided.

Sleep apnea is a sleep breathing disorder that involves instances where breathing stops for more than a certain period of time (e.g., 10 seconds) during sleep. Sleep apnea may be classified into obstructive sleep apnea, which occurs when the oral airway is blocked despite an attempt to breathe, and central sleep apnea in which the effort to breathe itself temporarily stops. Hypopnea is breathing that is reduced to half of the normal respiration volume or less, instead of completely stopping breathing, causing a drop in the concentration of oxygen in the blood.

### DISCLOSURE

### Technical Problem

A sleep breathing disorder (apnea or hypopnea) is a condition that many people have but are not aware of and can be a long-term health risk if left untreated.

Examples of diagnostic criteria for a sleep breathing disorder are as follows.

For example, an obstructive sleep breathing disorder may be diagnosed when two or more of the five symptoms, such as daytime somnolence, shortness of breath during sleep, not feeling refreshed after sleep, and loss of concentration due to daytime fatigue, are present and when a sleep breathing disorder is found on polysomnography (if apnea or hypopnea occurs 5 times or more per hour).

The severity of the obstructive sleep breathing disorder may be defined as mild when apnea-hypopnea occurs 5 times to less than 15 times per hour on polysomnography, moderate when 15 to less than 30 times per hour, and severe when times or more per hour.

Diagnosis of a sleep breathing disorder may require a visit to a specialized institution (e.g., a hospital or sleep center) or polysomnography using specialized devices (e.g., medical devices). This may impose burdens on the patient in terms of time, space, and expense.

Despite the high prevalence (e.g., 27% for male and 16% for female) for people over 40 years of age, most of them are often unaware of their sleep breathing disorder, which makes it difficult to seek appropriate treatment or correction.

Electronic devices are provided which may monitor breathing during periods of sleep. However, false alarms occur frequently due to inaccurate monitoring of a sleep breathing disorder, which may cause user discomfort or the user may fail to properly recognize the risk of a sleep breathing disorder.

### Technical Solution

Various embodiments disclosed in this document may provide an electronic device that eliminates factors that may cause mis-determination or false alarms when monitoring a sleep breathing disorder, thereby providing a relatively accurate monitoring result to a user at an appropriate time or lowering a false alarm rate, and an operation method thereof.

Various embodiments disclosed in this document may provide an electronic device capable of improving monitoring accuracy by adaptively varying a criterion for notification of a sleep breathing disorder according to an individual user, and an operation method thereof.

Various embodiments disclosed in this document may provide an electronic device capable of monitoring a sleep breathing disorder, of which is difficult for the user to be aware, using a portable or mobile electronic device that can be easily accessed in daily life, and capable of conveniently informing a user of a sleep breathing disorder, and an operation method thereof.

According to one or more non-limiting embodiments, an electronic device according to various embodiments may include a communication circuit, at least one sensor, and at least one processor connected to the communication circuit and the at least one sensor. The at least one processor may be configured to obtain biometric information of a user through the communication circuit or the at least one sensor during the user's sleep, determine a reference condition for detecting a sleep breathing disorder event, based on underlying disease information of the user, detect a sleep breathing disorder event, based on the biometric information and the reference condition, and provide a user interface for the sleep breathing disorder event as the sleep breathing disorder event is detected.

An operation method of an electronic device according to various embodiments may include obtaining biometric information of a user during the user's sleep, determining a reference condition for detecting a sleep breathing disorder event, based on underlying disease information of the user, detecting a sleep breathing disorder event, based on the biometric information and the reference condition, and providing a user interface for the sleep breathing disorder event as the sleep breathing disorder event is detected.

### Advantageous Effects

According to various embodiments, it is possible to monitor a sleep breathing disorder that is difficult to be aware of using a portable or mobile electronic device that can be easily accessed in daily life and easily notify the user of the sleep breathing disorder.

According to various embodiments, it is possible to provide a relatively accurate monitoring result to a user at an appropriate time or lower a false alarm rate by excluding a factor that may cause mis-determination when notifying of a sleep breathing disorder.

According to various embodiments, it is possible to provide an electronic device capable of improving monitoring accuracy by adaptively varying a criterion for notification of a sleep breathing disorder according to an individual user, and an operation method thereof.

In addition, various effects directly or indirectly recognized through this document may be provided.

### DESCRIPTION OF DRAWINGS

FIG. 1 is a block diagram of an electronic device in a network environment, according to various embodiments;

FIG. 2 is a block diagram of an electronic device according to an embodiment.

FIG. 3 is a block diagram illustrating configuration for each module of an electronic device according to an embodiment.

FIG. 4 is a flowchart illustrating an operation method of an electronic device according to an embodiment.

FIG. 5 is a flowchart illustrating an operation method of an electronic device according to an embodiment.

FIGS. 6A and 6B are diagrams illustrating a method of measuring oxygen saturation in an electronic device according to an embodiment.

FIG. 7 is an example of a user interface related to a sleep breathing disorder notification function of an electronic device according to an embodiment.

FIG. 8 is another example of a user interface related to a sleep breathing disorder notification function of an electronic device according to an embodiment.

FIG. 9 is another example of a user interface related to a sleep breathing disorder notification function of an electronic device according to an embodiment.

FIG. 10 is another example of a user interface related to a breathing disorder notification function for each sleep module of an electronic device according to an embodiment.

### MODE FOR INVENTION

Hereinafter, various embodiments will be described with reference to the accompanying drawings.

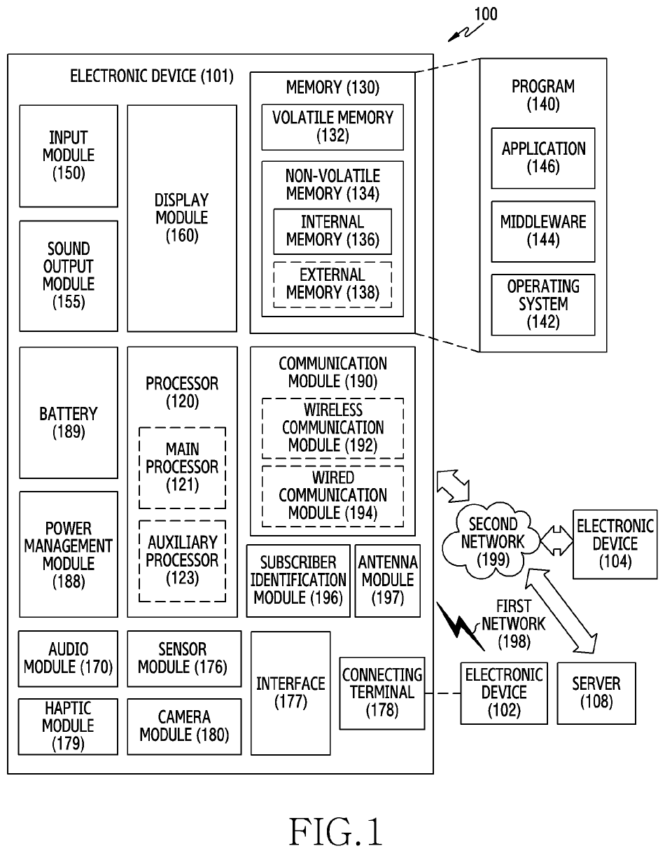

FIG. 1 is a block diagram illustrating an electronic device 101 in a network environment 100 according to various embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thererto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

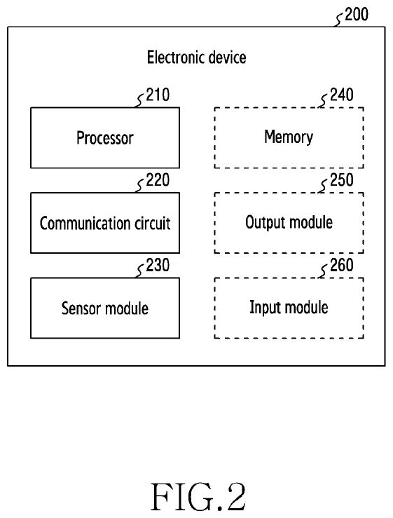

FIG. 2 is a block diagram of an electronic device according to an embodiment.

An electronic device 200 according to an embodiment may be intended to provide a sleep monitoring function. An electronic device 200 according to an embodiment may be intended to provide a sleep breathing disorder notification function. For example, the electronic device 200 may be implemented in the form of a wearable device (e.g., a smart watch, a smart ring, a smart band, or smart socks). The wearable device may be a device having a weight or battery capacity that the user is able to use without any difficulty in daily life or while sleeping. As another example, the electronic device 200 may be implemented in the form of a mobile device (e.g., a smartphone, a flexible smartphone, or a tablet).

Referring to FIG. 2, the electronic device 200 according to an embodiment may include a processor 210, a communication circuit 220, and/or a sensor module 230. The electronic device 200 may further include one or more of a memory 240, an output module 250, and an input module 260. The electronic device 200 may exclude at least one of the elements or may further include other elements.

For example, the electronic device 200 may correspond to the electronic device 101 in FIG. 1 or include at least some of the elements of the electronic device 101. At least some of the processor 210, the communication circuit 220, the sensor module 230, the memory 240, the output module 250, and/or the input module 260 included in the electronic device 200 may be electrically and/or operatively connected to each other, thereby exchanging signals (e.g., commands or data) with each other.

The electronic device 200 may include at least a part of the electronic device 101 illustrated in FIG. 1. For example, the processor 210 may correspond to the processor 120 (one of 121 or 123) in FIG. 1. The communication circuit 220 may include the communication module 190 in FIG. 1. The sensor module 230 may correspond to the sensor module 176 in FIG. 1 or include a part of the same. The memory 240 may include at least a part of the memory 130 in FIG. 1. The output module 250 may include at least some of the display module 160, the audio module 170, the sound output module 155, and the haptic module 179 in FIG. 1. The input module 260 may include at least a part (e.g., a microphone) of the input module 150 in FIG. 1.

The processor 210 may execute and/or control various functions supported by the electronic device 200. The processor 210 may control at least some of the communication circuit 220, the sensor module 230, the memory 240, the output module 250, and the input module 260. The processor 210 may execute an application and control a variety of hardware by executing codes or instructions written in a programming language stored in the memory 240 of the electronic device 200. For example, the processor 210 may execute an application (e.g., a sleep care application or a healthcare application) and provide a sleep monitoring function (or a sleep breathing disorder notification function) using the application. An application executed in the electronic device 200 may operate independently or in conjunction with an external electronic device (e.g., a user's smartphone) or the server 108.

As the instructions stored in the memory 240 are executed, the processor 210 may operate.

In an embodiment, the sensor module 230 may include at least one sensor.

For example, each sensor in the sensor module 230 may be one of a biometric sensor (e.g., an electrode sensor, a photoplethysmogram (PPG) sensor, an electrocardiography (ECG) sensor, a galvanic skin response (GSR) sensor, or a bioelectrical impedance analysis (BIA) sensor), or a motion sensor. The biometric sensor may measure a user's biometric information (e.g., heart activity information such as electrocardiogram and pulse, oxygen saturation, blood pressure, and blood sugar). The motion sensor may include an acceleration sensor, a gyro sensor, or a hybrid sensor (e.g., a 6-axis sensor) that is a combination thereof. However, the disclosure is not limited thereto, and the motion sensor may include any type of sensor capable of measuring motion information according to the user's motion (e.g., tossing and turning or moving) (or the movement of the electronic device 200 worn on the user).

The sensor module 230 may output at least one piece of biometric information of the user or motion information on the user's motion (or the movement of the electronic device 200).

In an embodiment, the communication circuit 220 may include a wireless communication module (e.g., the wireless communication module 192 in FIG. 1 (e.g., a cellular communication module, a short-range wireless communication module, or a global navigation satellite system (GNSS) communication module)). The communication circuit 220 may establish a communication connection with one or more external electronic devices (e.g., the electronic device 104 or the server 108 in FIG. 1) and transmit/receive various types of information or data. For example, the communication circuit 220 may support at least one of short-range wireless communication (e.g., Bluetooth, Bluetooth low energy (BLE), near-field communication (NFC), wireless fidelity (Wi-Fi) Direct, or infrared data association (IrDA)) and/or a long-range wireless communication (e.g., cellular communication, 5G communication, the Internet, or GNSS communication).

In an embodiment, the communication circuit 220 may support short-range wireless communication connection of the electronic device 200. For example, the communication circuit 220 may support a short-range wireless communication (e.g., Bluetooth, BLE, NFC, Wi-Fi Direct, or IrDA) between the electronic device 200 and an external electronic device (e.g., a user's smartphone or a wearable device worn on the user while sleeping).

In an embodiment, the communication circuit 220 may support a long-range wireless communication connection of the electronic device 200. For example, the communication circuit 220 may receive at least one piece of positioning information and location information through long-range wireless communication and transmit the same to the processor 210. For example, the communication circuit 220 may support a long-range wireless communication (e.g., cellular communication, 5G communication, or the Internet) connection between an external server (e.g., the server 108 in FIG. 1) and the electronic device 200, thereby receiving information about the user's current location and/or place from the external server. As another example, the communication circuit 220 may support a GNSS communication connection between a satellite and the electronic device 200 for positioning the electronic device 200, thereby receiving information about the user's current location and/or place from the satellite.

In an embodiment, the output module 250 may include one or more modules for providing a user interface. For example, the output module 250 may include one or more of a display (e.g., the display module 160 in FIG. 1), an audio module (e.g., the audio module 170), a sound output module (e.g., the sound output module 155), and a haptic module (e.g., the haptic module 179).

In an embodiment, the processor 210 may execute a sleep monitoring function (or a sleep breathing disorder notification function).

In an embodiment, the processor 210 may detect the user's sleep. For example, the processor 210 may detect whether or not the user is sleeping through at least one sensor (e.g., a biometric sensor and/or a motion sensor). The at least one sensor may be included in the sensor module 230 of the electronic device 200 or included in an external electronic device (e.g., a wearable device) connected to the electronic device 200 through short-range wireless communication.

For example, the processor 210 may automatically detect sleep onset/offset using at least one sensor (e.g., a motion sensor and/or a biometric sensor) in the sensor module 230 or detect sleep starting/ending by a user input.

For example, the processor 210 may detect at least one of a user's bedtime (or start of sleep), wake-up time (or end of sleep), or sleeping hours, based on at least one piece of the user's biometric information and motion information.

In an embodiment, the processor 210 may obtain the user's biometric information for monitoring a sleep breathing disorder while the user is sleeping. For example, the biometric information may include at least one of oxygen saturation, heart rate, respiration, blood pressure, blood sugar, and snoring. For example, the processor 210 may obtain information about oxygen saturation, heart rate, respiration, blood pressure, and/or blood sugar through a biometric sensor in the sensor module 230. As another example, the processor 210 may obtain information about the user's snoring sound through a microphone in the input module 260.

For example, the processor 210 may obtain biometric information while the user is sleeping through the sensor module 230 and/or the communication circuit 220. The processor 210 may collect biometric information during a total sleep period from bedtime to wake-up time or for a specified period of time (e.g., 4 hours).

For example, the processor 210 may obtain biometric information or motion information through the sensor module 230. As another example, the processor 210 may receive biometric information or motion information from an external electronic device (e.g., a wearable device worn on the user who is sleeping or a mobile device placed close to the user who is sleeping) through the communication circuit 220 (e.g., a short-range wireless communication module). As another example, the processor 210 may obtain (e.g., detect, process, or calculate) biometric information in sleep from information measured through the sensor module 230 or information received through the communication circuit 220 (e.g., a short-range wireless communication module).

In an embodiment, the processor 210 may determine a reference condition for detecting a sleep breathing disorder event, based on underlying disease information of the user.

The underlying disease information may include information about the presence or absence of an underlying disease and/or an underlying disease state. For example, the underlying disease information may include information about at least one of hypertension, diabetes (or high blood sugar), obesity (or overweight), alcoholism, caffeine addiction, chronic stress, or sleep posture disorder.

The user's underlying disease information may be identified or obtained in various ways. For example, the user's underlying disease information may be identified by a method of collecting (or processing) the information by itself through the sensor module 230, a method of collecting (or extracting) the information from an external electronic device (e.g., the server 108 in FIG. 1, a sleep care provider server, a medical service servers, or a home server) through the communication circuit 220, a method of pre-storing the information in the memory 240 or calculating pre-stored data, or a combination of the above methods. However, the disclosure is not limited thereto.

In an embodiment, the processor 210 of the electronic device 200 (e.g., a user's wearable device) may directly measure the user's biometric information through the sensor module 230 or receive the user's biometric information measured by the external electronic device 200 (e.g., a user's smartphone) through the communication circuit 220.

In an embodiment, the processor 210 may monitor the respiration of the user in a sleep state using the user's biometric information obtained through the sensor module 230 and/or the communication circuit 220. The processor 210 may evaluate the presence or absence of a sleep breathing disorder and/or the degree (or risk) of a sleep breathing disorder by the monitoring. An apnea-hypopnea index (AHI) may be utilized as a monitoring reference. AHI may be an index indicating whether or not there is a sleep disorder called apnea (or hypopnea) and how severe it is. AHI is also a factor indicating the risk of a sleep breathing disorder, and may indicate the number of times apnea or hypopnea occurs per hour.

A reference condition (e.g., an AHI range condition) for monitoring a sleep breathing disorder may vary depending on whether or not the user has an underlying disease and/or on the underlying disease state (e.g., obesity, blood sugar, blood pressure, etc.). The processor 210 may change the reference condition for monitoring a sleep breathing disorder depending on the user's underlying disease state, thereby increasing the accuracy and/or sensitivity of monitoring and lowering the false alarm rate.

For example, even with the same AHI value detected from the user's biometric information, the reference condition for monitoring a sleep breathing disorder may vary depending on the underlying disease state (e.g., overweight, hypertension, or hyperglycemia) of the user.

In an embodiment, the processor 210 may detect a sleep breathing disorder event, based on the user's biometric information and reference condition.

In an embodiment, the processor 210 may detect an AHI value (or current AHI value) from the user's biometric information (e.g., information about at least one of oxygen saturation, heart rate, heart rate variability, blood sugar, and snoring) and detect a sleep breathing disorder event, based on whether or not the detected AHI value exceeds a AHI threshold value according to a reference condition.

In an embodiment, the reference condition for detecting the sleep breathing disorder event may include an AHI range condition. According to a non-limiting embodiment, it may be detected whether or not a sleep breathing disorder event occurs, based on the AHI threshold value.

In an embodiment, the processor 210 may adjust (or change) the reference condition (e.g., the AHI threshold value) for detecting a sleep breathing disorder event from a predetermined first condition to a second condition, based on the user's underlying disease information.

In an embodiment, the reference condition for detecting a sleep breathing disorder event may include an AHI range condition.

For example, the operation of adjusting the reference condition may be an operation of changing the AHI threshold value for detecting the occurrence of a sleep breathing disorder event from a first value, which is a default value, to a second value in consideration of whether or not the user has an underlying disease and/or the underlying disease state.

In an embodiment, the electronic device 200 may adjust the reference condition depending on whether or not the user has an underlying disease. The electronic device 200 may identify whether or not the user has an underlying disease from the user's underlying disease information. The electronic device 200 may adjust the reference condition for detecting a sleep breathing disorder event from a predetermined first condition to a second condition, based on whether or not the user has an underlying disease.

In an embodiment, the processor 210 may adjust the reference condition (e.g., an AHI threshold value) for detecting a sleep breathing disorder event, based on a risk level according to the user's underlying disease state. The electronic device 200 may determine a risk level of a sleep breathing disorder, based on the user's underlying disease information, and adjust the reference condition from a predetermined first condition to a second condition, based on the determined risk level. In an embodiment, as a sleep breathing disorder event is detected, the processor 210 may provide a user interface for the sleep breathing disorder event.

The user interface for a sleep breathing disorder may be provided through at least one of the output module 250 of the electronic device 200 (e.g., a user's wearable device) and/or an external electronic device (e.g., a user's smartphone) connected to the electronic device 200 through short-range wireless communication.

For example, the electronic device 200 (e.g., a wearable device such as a smart watch) may autonomously output a user interface for a sleep breathing disorder event through the output module 250. As another example, the electronic device 200 may transmit information about a user interface for a sleep breathing disorder event to an external electronic device (e.g., a wearable device worn on the user who is sleeping or a mobile device placed close to the user who is sleeping) through the communication circuit 220 (e.g., a short-range wireless communication module) such that the external electronic device may output the user interface. As another example, the electronic device 200 may provide a user interface for a sleep breathing disorder event substantially simultaneously through the output module 250 and an external electronic device.

The user interface for a sleep breathing disorder event may be intended for notification of a sleep breathing disorder event. The user interface may be implemented as a visual type (e.g., a screen or notification messages), an auditory type (e.g., audio signals such as music or sound), a tactile type (e.g., vibration), or a hybrid type combining at least some of them.

For example, a screen or a notification message for a sleep breathing disorder event may be displayed on the display of the electronic device 200. As another example, an audio signal for a sleep breathing disorder event (e.g., a warning sound, a notification voice, a specified music, or a sound of a specified pattern) may be output through the audio module of the electronic device 200. As another example, vibration may be output through the haptic module of the electronic device 200. As another example, a user interface (e.g., at least one of a screen, audio signal, and vibration) may be output through the electronic device 200 (e.g., a smart watch) and, at the same time, the corresponding user interface (e.g., at least one of a screen, audio signal, and vibration) may be output through an external electronic device (e.g., a smartphone) connected to the electronic device 200 through short-range wireless communication.

In an embodiment, the processor 210 may determine whether or not to deactivate the sleep breathing disorder notification function, based on a user's temporary state information. The temporary state information may be information about environmental and/or physiological factors that may cause mis-determination of the sleep breathing disorder state. For example, the temporary state information may include information about at least one of alcohol intake, caffeine intake, stress level, lifestyle (e.g., average sleep duration, bedtime, and wake-up time), location, and place. The processor 210 may deactivate the sleep breathing disorder notification function, based on the determination. For example, if the sleep breathing disorder notification function is deactivated (or turned oft), the entire sleep monitoring function may be turned off, or only the notification function in the sleep monitoring function may be selectively turned off. As the sleep breathing disorder notification function is deactivated, the electronic device 200 may skip (or omit) at least one of the operation of adjusting a reference condition, the operation of detecting a sleep breathing disorder event, and/or the operation of providing a user interface.

In an embodiment, the processor 210 may detect a sleep breathing disorder event by further considering the user's previous sleep record. For example, the user's previous sleep record may include at least one piece of first sleep data about the user's past sleep activity repeated a specified number of times (e.g., 7 or 30 times) or more, second sleep data about the user's past sleep activity for a recent predetermined period (e.g., a week or a month), and sleep breathing disorder analysis result data based on the first sleep data or the second sleep data.

In an embodiment, the electronic device 200 may adaptively change a reference condition (e.g., an AHI threshold value) for detecting a sleep breathing disorder event depending on a user's underlying disease state or a change in the underlying disease state and perform monitoring of a sleep breathing disorder using the changed reference condition. The presence or absence of a sleep breathing disorder and/or the risk of a sleep breathing disorder may be evaluated by the monitoring.

For example, the processor according to a non-limiting embodiment can determine an underlying disease state of a user, and determine a reference condition such as an AHI threshold value, for example, based at least in part on the underlying disease state, and can actively or adaptively change the AHI threshold value in response to detecting a change in the underlying disease state. The at least one sensor can monitor biometric information of the user during a period of sleep, and the processor can assign an AHI value to the biometric information. Accordingly, the processor can detect a sleep breathing disorder event in response to the AHI value exceeding the changed AHI threshold value, and control the electronic device to generate an alert using, for example, a user interface, an auditory alert (e.g., audio signals such as music or sound), and/or a tactile alert (e.g., vibration) to indicate the detected sleep breathing disorder event.

In an embodiment, the processor 210 of the electronic device 200 may identify the user as one of either an attention-required user who has an underlying disease and a general user who has no underlying disease, based on the user's underlying disease information. A disease state may include, for example, a hypertensive patient, a diabetic patient, a user who is overweight or medically obese, a user whose stress level exceeds a specified threshold value, and a user whose average sleep duration is less than or equal to a threshold value may be the attention-required users. Users other than the attention-required users may be the general users. If the user is a general user, the processor 210 may maintain the reference condition as a first condition without adjustment. If the user is an attention-required user, the processor 210 may adjust the reference condition (e.g., the AHI threshold value) from a preset or preconfigured first condition to a second condition. For example, the electronic device 200 may recognize whether or not the user has an underlying disease through the user's biometric information (e.g., oxygen saturation, blood pressure, blood sugar, and BIA data) and change the reference condition (e.g., an AHI threshold condition) for detecting a sleep breathing disorder event according to the result.

For example, if the user is a general user who has no underlying disease, the processor 210 may maintain an AHI threshold value included in the preconfigured reference condition to be a first value without adjustment. Based on the case where an AHI value obtained from the biometric information exceeds the first value, the processor 210 may detect a sleep breathing disorder event of a general user. If the user is an attention-required user, the processor 210 may lower the AHI threshold value included in the preconfigured reference condition from the first value to a second value. Based on the case where an AHI value (or current AHI value) obtained from the biometric information exceeds the second value, the processor 210 may detect a sleep breathing disorder event of an attention-required user.

For example, in the case of a general user who has no underlying disease, the AHI threshold value for detecting a sleep breathing disorder event may be a default value of 15. In the case of an attention-required user who has an underlying disease (e.g., hypertension, diabetes, or obesity), the AHI threshold value for detecting a sleep breathing disorder event may be lowered to 5. In the case where a unit event in which the AHI value detected from the user's biometric information exceeds a threshold value of 5 occurs (or if a unit event in which the AHI value exceeds the threshold value occurs three times or more during the last week), the electronic device 200 may determine that a sleep breathing disorder event has occurred and provide a notification informing the user of the occurrence of a sleep breathing disorder.

In an embodiment, the processor 210 may adjust the AHI threshold value in stages according to the risk level of the underlying disease. For example, a user who has no underlying disease may be classified as a first user with the lowest risk level, a user who has an underlying disease with a low severity may be classified as a second user with an intermediate risk level, and a user who has an underlying disease and a high severity may be classified as a third user with the highest risk level. For the first user, the AHI threshold value may be maintained to be a first value, and a sleep breathing disorder event may be detected based on the case where a current AHI value detected from the biometric information exceeds the first value. For the second user, the AHI threshold value may be lowered to a second value lower than the first value, and a sleep breathing disorder event may be detected based on the case where the current AHI value exceeds the second value. For the third user, the AHI threshold value may be lowered from the first value to a third value, which is lower than the second value, and a sleep breathing disorder event may be detected based on the case where the current AHI value exceeds the third value.

In an embodiment, the user's underlying disease information may be updated.

For example, the processor 210 may update prestored underlying disease information (e.g., information about the presence or absence of an underlying disease and/or the underlying disease state) using biometric information in user's sleep, and based on the updated underlying disease information, determine a reference condition for monitoring a sleep breathing disorder. Accordingly, information about whether or not the user has an underlying disease and/or the underlying disease state may be updated in real time during sleep, thereby allowing for the processor to adaptively change the reference condition (e.g., the AHI threshold value) and improve monitoring accuracy.

As another example, the user's underlying disease information may be changed periodically (e.g., every specified day of the month) and/or when an update event occurs (e.g., at the end of sleep, upon user input, upon visiting hospital, and/or upon inputting medical results). The processor 210 may adjust the reference condition as the user's underlying disease information is updated, and detect a sleep breathing disorder event, based on the adjusted reference condition.

In an embodiment, the electronic device 200 may adaptively change a reference condition (e.g., an AHI threshold condition) for detecting a sleep breathing disorder event depending on whether or not the user has an underlying disease and/or a change in the underlying disease state.

For example, if the user changes from a general user to the state of having an underlying disease, an AHI range for monitoring a sleep breathing disorder may be lowered (e.g., the AHI threshold value is lowered from 15 to 5, and a notification is provided if the AHI value is 5 or more), thereby increasing the sensitivity of monitoring. If the underlying disease is improved due to improvement of a user's lifestyle, the AHI range may be re-adjusted (e.g., the AHI threshold value is increased from 5 to 15, and a notification is provided if the AHI value is 15 or more).

Unlike polysomnography performed in a specialized place such as a hospital, since the electronic device 200 (e.g., a smartphone or a wearable device) is always carried by the user or worn on the body, sleep monitoring or long-term sleep monitoring is possible in a daily environment.

If oxygen supply is not enough during sleep due to a sleep breathing disorder, chronic fatigue may occur, which may lead to fatal biological damage (e.g., cardiovascular disease or stroke).

According to an embodiment, providing a function of monitoring a sleep breathing disorder by the electronic device 200 that the user always carries or wears may help the user to properly cope with a sleep breathing disorder that is difficult to recognize and may cause fatal damage.

The sleep breathing disorder may be more fatal to people with an underlying disease. For example, a person having an underlying disease may be classified as an attention-required user who belongs to a fatal risk group, and a person who has no underlying disease may be classified as a general user.

According to an embodiment, by adjusting the reference condition for monitoring a sleep breathing disorder depending on whether or not the user has an underlying disease, a person with an underlying disease may not miss a sleep breathing disorder notification, and a person without an underlying disease may receive less false alarms related to a sleep breathing disorder.

In addition, the possibility (or probability or risk) of suffering fatal biological damage due to a sleep breathing disorder may vary depending on the user's underlying disease state (or chronic disease state).

According to an embodiment, a condition for monitoring a sleep breathing disorder may be personalized to the user by applying a risk level depending on the user's underlying disease state.

According to an embodiment, a condition for monitoring a sleep breathing disorder may be adaptively adjusted by appropriately reflecting the possibility (or probability or risk) of damage due to a sleep breathing disorder or learning the user's previous sleep record using the user's underlying disease information.

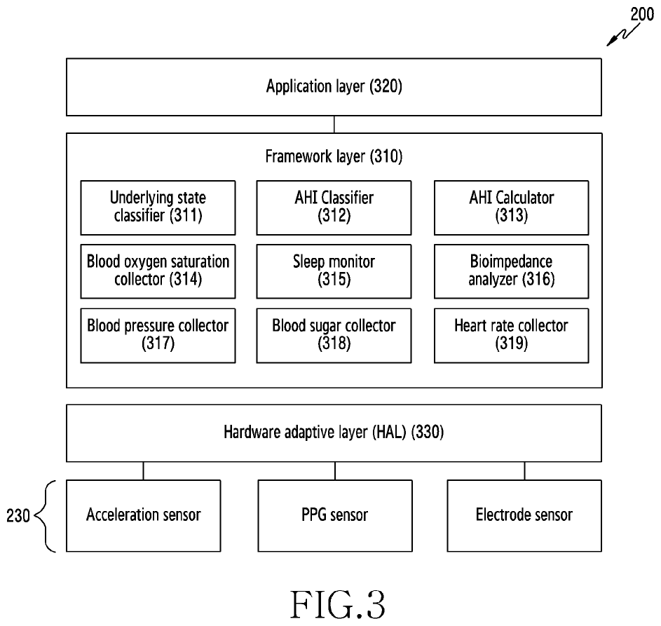

FIG. 3 is a block diagram illustrating configuration for each module of an electronic device according to an embodiment.

Referring to FIG. 3, the electronic device 200 may include a hardware layer including a sensor module 230 (e.g., an acceleration sensor, a PPG sensor, and an electrode sensor), a hardware adaptive layer (HAL) 330, a framework layer 310, and an application layer 320.

In an embodiment, the hardware layer may include a sensor module 230 (e.g., an acceleration sensor, a PPG sensor, and an electrode sensor). Each element of the hardware layer may be implemented as physically separate hardware.

Respective elements of the hardware adaptive layer 330, the framework layer 310, and/or the application layer 320 may be implemented as firmware and/or software and stored in the memory 240 of the electronic device 200. For example, the processor 210 of the electronic device 200 may execute instructions stored in the memory 240 to operate the respective elements. Alternatively, the respective elements may operate as the instructions stored in the memory 240 are executed.

A user's biometric information and/or motion information measured through the sensor module 230 (e.g., an acceleration sensor, a PPG sensor, and an electrode sensor) may be transmitted to the framework layer 310 and/or the application layer 320 through the hardware adaptive layer 330.

The application layer 320 may include one or more applications (e.g., a healthcare application and a sleep care application).

In an embodiment, the framework layer 310 may include at least a part of an underlying state classifier 311, an AHI classifier 312, an AHI calculator 313, a blood oxygen saturation collector 314, a sleep monitor 315, and a bioimpedance analyzer 316, a blood pressure collector 317, a blood sugar collector 318, or a heart rate collector 319.

The sleep monitor 315 may measure motion information through an acceleration sensor in the sensor module 230, for example, and detect sleep starting/ending using the motion information to start/stop monitoring of a sleep breathing disorder. The monitoring of a sleep breathing disorder may be performed during a sleep period from the start time of sleep to the end time of sleep.

The blood oxygen saturation collector 314 may measure the oxygen saturation in the blood during a sleep period through a PPG sensor or an electrode sensor in the sensor module 230. The blood pressure collector 317 may measure blood pressure during a sleep period through the PPG sensor or the electrode sensor. The blood sugar collector 318 may measure blood sugar during a sleep period through the PPG sensor or the electrode sensor. The heart rate collector 319 may measure a heart rate or data on heart rate variability during a sleep period through the PPG sensor or the electrode sensor. The bioimpedance analyzer 316 may measure BIA data during a sleep period through a BIA sensor (not shown) in the sensor module 230.

The AHI calculator 313 may calculate an AHI value by analyzing biometric information (e.g., oxygen saturation) detected during a sleep period and assign the AHI value to the biometric information obtaining while monitoring the user during a period of sleep.

The underlying state classifier 311 may collect and a user's biometric information (e.g., blood pressure, blood sugar, oxygen saturation, heart rate, heart rate variability, and BIA data) during a sleep period through the PPG sensor or the electrode sensor in the sensor module 230 and, based on the obtained biometric information, classify the user (or user type) as one of either an attention-required user (e.g., a user having an underlying disease or an obese user) or a general user.

The AHI classifier 312 may classify an AHI range condition (or AHI threshold condition) for detecting a sleep breathing disorder event depending on a user type. For example, if the user is a general user, the AHI range condition may be classified as a first condition corresponds to “the case where an AHI value exceeds 15”. If the user is an attention-required user, the AHI range condition may be classified as a second condition corresponding to “the case where the AHI value exceeds 5”.

Applications (e.g., a healthcare application and a sleep care application) in the application layer 320 may perform monitoring a sleep breathing disorder by interworking with the framework layer 310 and provide the user with a notification indicating the occurrence of a sleep breathing disorder according to the monitoring result. A reference condition for monitoring a sleep breathing disorder may include an AHI range condition (or an AHI threshold condition). The reference condition may be adaptively changed depending on the user's underlying state (e.g., the presence or absence of an underlying disease and/or an abnormal or normal physical state).

For example, the application may receive information about a user type (e.g., any one of an attention-required user and a general user) from the underlying state classifier 311, receive a user's AHI value (or current AHI value) from the AHI calculator 313, and receive information about an AHI range condition (or AHI threshold condition) for detecting a sleep breathing disorder event from the AHI classifier 312.

If the user is an attention-required user, the application may apply a second condition for an attention-required user to the AHI range condition, thereby monitoring whether or not a sleep breathing disorder occurs and/or a risk level thereof, and provide a notification according to the monitoring result. If the user is a general user, the application may apply a second condition for a general user to the AHI range condition, thereby monitoring whether or not a sleep breathing disorder occurs and/or a risk level thereof, and provide a notification according to the monitoring result.

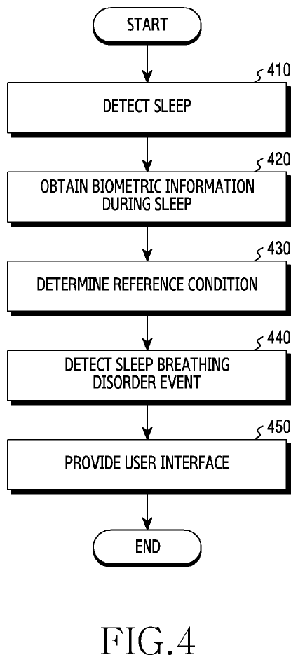

FIG. 4 is a flowchart illustrating an operation method of an electronic device according to an embodiment.

Although the method in FIG. 4 is assumed to be performed by the electronic device 200 in FIG. 2 for convenience, the disclosure is not limited thereto. For example, the method in FIG. 4 may be performed by an electronic device (e.g., the processor 210 in FIG. 2, an application (e.g., a sleep care application) executed in the electronic device 200 in FIG. 2, or the electronic device 101 or the processor 120 in FIG. 1). In some embodiments, some of the operations in the method shown in FIG. 4 may be omitted or integrated, the sequence of some operations may change, or other operations may be added thereto.

For example, the electronic device 200 (e.g., a wearable device such as a smart watch or a smart ring) may be worn on the user during a sleep activity. The electronic device 200 (e.g., a user's wearable device) may be connected to at least one external electronic device (e.g., a user's smartphone) through short-range wireless communication to interwork with each other.

Referring to FIG. 4, in operation 410, the electronic device 200 may detect the user's sleep. For example, the electronic device 200 may detect whether or not the user is sleeping through at least one sensor (e.g., a biometric sensor and/or a motion sensor). The at least one sensor may be included in the electronic device 200 or may be included in an external electronic device (e.g., a wearable device) connected to the electronic device 200 through short-range wireless communication.

As an example, the electronic device 200 may measure motion information according to the user's lying posture or whether or not the user moves through a motion sensor (e.g., an acceleration sensor) and determine whether or not the user is sleeping, based on the motion information.

As another example, the electronic device 200 may measure a heart rate or heart rate variability (HRV) using a biometric sensor (e.g., a PPG sensor) and determine whether or not the user is sleeping, based on the measurement result. For example, the electronic device 200 may extract at least one of an LF value indicating the degree of sympathetic activity (power-in-low frequency range), an HF value indicating the degree of parasympathetic activity (power-in-high frequency), normalized LF and HF values, and/or an LF/HF value (ratio of LF to HF) indicating the overall balance of the autonomic nervous system through frequency analysis of heart rate information measured through the PPG sensor, and estimate whether or not the user is sleeping or a sleep stage (e.g., a light sleep stage, a deep sleep stage, and or a rapid eye movement (REM) sleep stage) using the extracted value.

In operation 420, the electronic device 200 may obtain biometric information of the user while the user is sleeping for monitoring sleep (or a sleep breathing disorder). For example, the electronic device 200 may measure biometric information through at least one sensor (e.g., a biometric sensor in the sensor module 230) or receive biometric information measured by an external electronic device (e.g., a wearable device) through the communication circuit 220.

For example, the electronic device 200 may collect biometric information about at least one of oxygen saturation, heart rate, respiration, blood pressure, blood sugar, and snoring.

The operation of obtaining biometric information in sleep may be continuously performed for a specified time or may be periodically repeated.

In an embodiment, the electronic device 200 may measure oxygen saturation (SpO2) during sleep using a biometric sensor (e.g., a PPG sensor) for monitoring a sleep breathing disorder. For example, the biometric sensor may irradiate the user's skin with red light and infrared light, and estimate oxygen saturation, which is blood oxygen concentration, based on the difference in absorbance between hemoglobin in a blood vessel and oxyhemoglobin.

Oxygen saturation may be measured even in an awake state (or in a usual or non-sleep state), and it may be preferable to measure the oxygen saturation in the state in which the user move less, such as in a sleep state, due to dynamic noise. In the case where oxygen saturation is measured in a sleep state and used for monitoring a sleep breathing disorder, the reliability of the measurement result may be improved and accurate monitoring may be possible.

However, the biometric information collected for monitoring a sleep breathing disorder is not limited to oxygen saturation. In addition to oxygen saturation, the electronic device 200 may collect various types of biometric information (e.g., information about at least one of heart rate, respiration, blood pressure, blood sugar, and snoring) in various ways.

For example, the electronic device 200 may measure a heart rate or heart rate variability during sleep.

As another example, the electronic device 200 may measure blood pressure during sleep. For example, the blood pressure may be estimated by a pulse wave analysis (PWA) method. In this case, a systolic blood pressure (SBP) and a diastolic blood pressure (DBP) may be estimated from a waveform of a signal derived by ensemble-averaging pulse wave information.

As another example, the electronic device 200 may measure blood glucose during sleep. Blood glucose indicates the concentration of glucose in the blood and may be measured in various ways. For example, the electronic device 200 may irradiate the user's skin with light of a specified wavelength band through a biometric sensor (e.g., a PPG sensor) and detect energy generated by the reaction of glucose, thereby estimating the blood glucose level.

In some embodiments, operation 410 for detecting whether or not the user is sleeping may be omitted, or operation 410 and operation 420 may be integrated into one.

For example, since the brain rests and the parasympathetic nervous system is activated to recover the body in the sleep state, compared to the awake state, even if the same type of biometric information is measured, the biometric information measured may be different between the awake state and the sleep state. For example, the oxygen saturation signal may have a different phase from the oxygen saturation signal measured in the awake state. The electronic device 200 may detect that the user is in the sleep state using biometric information collected during sleep without a separate operation.

In operation 430, the electronic device 200 may determine a reference condition for detecting a sleep breathing disorder event, based on the user's underlying disease information.

The underlying disease information may include information about the presence or absence of an underlying disease and/or an underlying disease state. For example, the underlying disease information may include information about at least one of hypertension, diabetes (or high blood sugar), obesity (or overweight), alcoholism, caffeine addiction, chronic stress, and sleep posture disorders.

In an embodiment, the electronic device 200 may obtain the user's underlying disease information and adjust a reference condition for detecting a sleep breathing disorder event, based on the underlying disease information.

The electronic device 200 may obtain underlying disease information in various ways. For example, the underlying disease information may be prestored, estimated in real time, input by the user, or received from an external electronic device (e.g., a server or a wearable device).

Examples of a method of obtaining the underlying disease information are as follows.

For example, the underlying disease information may be manually input. The user may directly check items such as high blood pressure, blood sugar, and/or obesity. Alternatively, the underlying disease may be estimated through the blood pressure, blood sugar, and/or weight (body composition) input by the user.

As another example, information about a personal health record (PHR) and/or an electronic medical record (EMR) may be utilized. The information may be stored in a server of a company or public institution. The electronic device 200 may receive the corresponding information from the server through user authentication and store the same in the memory 240. The electronic device 200 may recognize whether or not the user has an underlying disease and information about a doctor's opinion or medical treatment (e.g., a sleep apnea diagnosis result) using the stored information.

As another example, the electronic device 200 may measure blood pressure, blood sugar, or BIA data through a wearable device connected through short-range wireless communication and monitor underlying diseases (or chronic diseases) such as hypertension, hyperglycemia, and/or obesity using the measured values.

As another example, the electronic device 200 may receive data from an external medical device (e.g., a cuff sphygmomanometer, an invasive glucometer, and/or a smart scale) connected through wireless communication and identify underlying disease information from the received data.

In an embodiment, the electronic device 200 may adjust a reference condition depending on whether or not the user has an underlying disease. The electronic device 200 may identify whether or not the user has an underlying disease from the user's underlying disease information. Based on whether or not the user has an underlying disease, the electronic device 200 may adjust a reference condition for detecting a sleep breathing disorder event from a preconfigured first condition to a second condition.

For example, based on the user's underlying disease information, the electronic device 200 may identify the user as one of either an attention-required user who has an underlying disease and a general user who has no underlying disease. For example, hypertensive patient, a diabetic patient, a user who is overweight, a user whose stress level exceeds a specified threshold value, and a user whose average sleep duration is less than or equal to a threshold value may be the attention-required users. Users other than the attention-required users may be the general users. If the user is an attention-required user, the electronic device 200 may adjust the reference condition from a preconfigured first condition to a second condition. If the user is a general user, the electronic device 200 may maintain the reference condition as a first condition without adjustment.

In an embodiment, the reference condition for monitoring a sleep breathing disorder may include an AHI range condition.

For example, the operation of adjusting the reference condition may be an operation of changing the AHI threshold value for detecting occurrence of a sleep breathing disorder event from a first value, which is a default value, to a second value in consideration of whether or not the user has an underlying disease and/or the underlying disease state.

For example, if the user has an underlying disease, the electronic device 200 may lower the AHI threshold value for detecting the occurrence of a sleep breathing disorder event from a first value, which is a default value, to a second value in consideration of underlying disease information. Based on the case where an AHI value (or current AHI value) obtained from the biometric information exceeds the second value, the electronic device 200 may detect a sleep breathing disorder event. If the AHI threshold value is lowered, a reference for monitoring a sleep breathing disorder may be strengthened and accurate monitoring may be possible. If the user does not have an underlying disease, the electronic device 200 may maintain the AHI threshold value included in the reference condition without adjustment.

In an embodiment, the electronic device 200 may adjust a reference condition (e.g., AHI threshold value) for detecting a sleep breathing disorder event, based on a risk level according to the user's underlying disease state. The electronic device 200 may determine a risk level of a sleep breathing disorder, based on the user's underlying disease information, and adjust the reference condition from a predetermined first condition to a second condition, based on the determined risk level.

For example, the electronic device 200 may lower the AHI threshold value included in the reference condition from the first value to a third value, based on the risk level. The electronic device 200 may detect whether or not a sleep breathing disorder event occurs based on the case where the AHI value (or current AHI value) obtained from the biometric information exceeds the third value.

In operation 440, the electronic device 200 may detect a sleep breathing disorder (apnea or hypopnea) event, based on the biometric information obtained in operation 420 and the reference condition determined in operation 430.

In an embodiment, the user's underlying disease information may be updated periodically and/or when an update event occurs (e.g., at the end of sleep, upon user input, upon visiting hospital, and/or upon inputting medical results). The electronic device 200 may actively adjust the reference condition, based on the updated underlying disease information, and detect a sleep breathing disorder event, based on the adjusted underlying disease information.

For example, the electronic device 200 may identify whether or not the user has an underlying disease or a change related to the underlying disease state from the updated underlying disease information, adjust the reference condition according to the identification result, and detect a sleep breathing disorder event, based on the adjusted reference condition. For example, if an underlying disease occurs in a user who does not have an underlying disease, the AHI threshold value for detecting a sleep breathing disorder event may be lowered from the first value to the second value. As another example, if the degree of underlying disease (e.g., blood pressure level and glucose level) is improved, the AHI threshold value may be increased from the third value to the second value.

In an embodiment, the electronic device 200 may detect a sleep breathing disorder event by further considering a user's previous sleep record. For example, the user's previous sleep record may include at least one piece of first sleep data about the user's past sleep activity repeated a specified number of times (e.g., 7 or 30 times) or more, second sleep data about the user's past sleep activity for a recent predetermined period (e.g., a week or a month), and sleep breathing disorder analysis result data based on the first sleep data or the second sleep data. For example, the reference condition for detecting a sleep breathing disorder event may include an AHI range condition. The reference condition may further include information about a unit event in the previous sleep record (e.g., the number of times an AHI value exceeds a threshold value in the user's previous sleep activity, and time or date thereof).

Since there are various factors in which the sleep breathing disorder state may be misjudged, in an embodiment, the electronic device 200 may monitor sleep for several days according to the user's previous sleep record to determine whether or not a sleep breathing disorder event occurs, thereby reducing the risk of mis-determination.

For example, even if a unit event in which the AHI value detected from the biometric information exceeds an AHI threshold value occurs, the electronic device 200 may determine that a sleep breathing disorder event has occurred only if the unit event in which the AHI value exceeds the threshold value continuously occurs again on the next day, instead of immediately determining that a sleep breathing disorder event has occurred.

As another example, if a unit event in which the AHI value exceeds an AHI threshold value occurs a specified number of times or more for a specified time (e.g., 2 times or more for 3 days within 10 days), the electronic device 200 may determine that a sleep breathing disorder event has occurred.

As another example, even if a unit event in which the AHI value exceeds an AHI threshold value occurs, the electronic device 200 may suspend the determination of whether or not a sleep breathing disorder event occurs or determine that a sleep breathing disorder event has not occurred in the case where there are various environmental and/or physiological factors. For example, if the user consumes alcohol, it may involve a temporary sleep breathing disorder, so the determination of whether or not a sleep breathing disorder event occurs may be suspended, and it may be determined whether or not a sleep breathing disorder event occurs when alcohol is not consumed. Alternatively, if the sleeping place is the outdoors or the place other than a usual place, the determination of whether or not a sleep breathing disorder event occurs may be suspended, or a sleep breathing disorder notification function may not be performed.

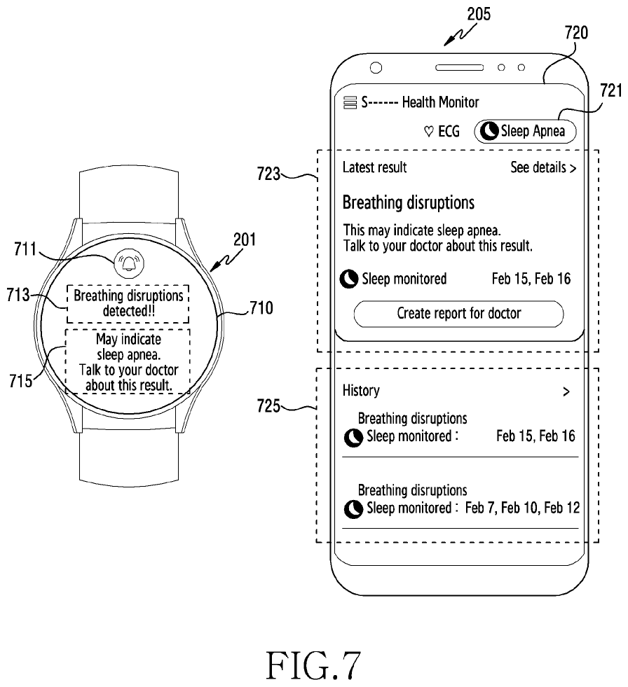

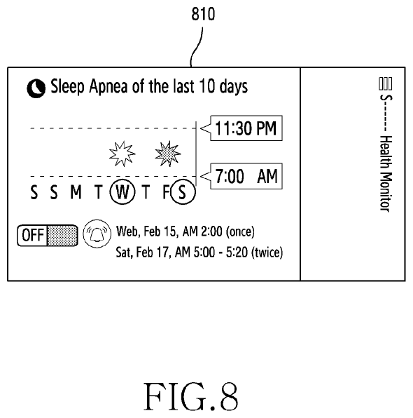

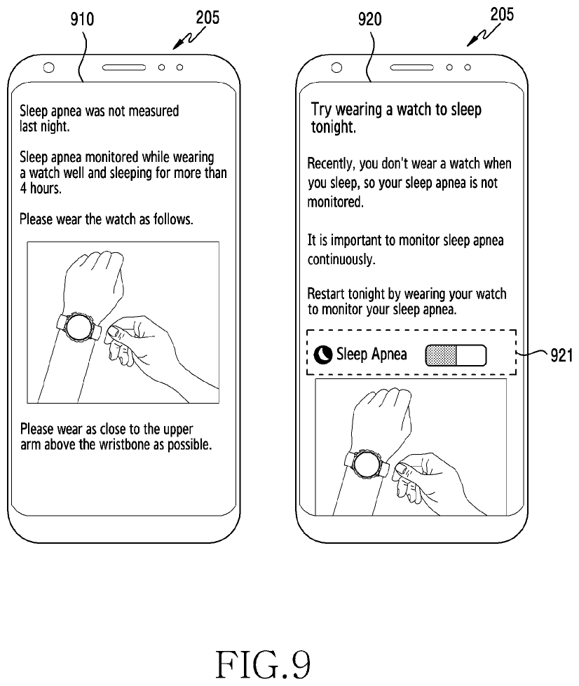

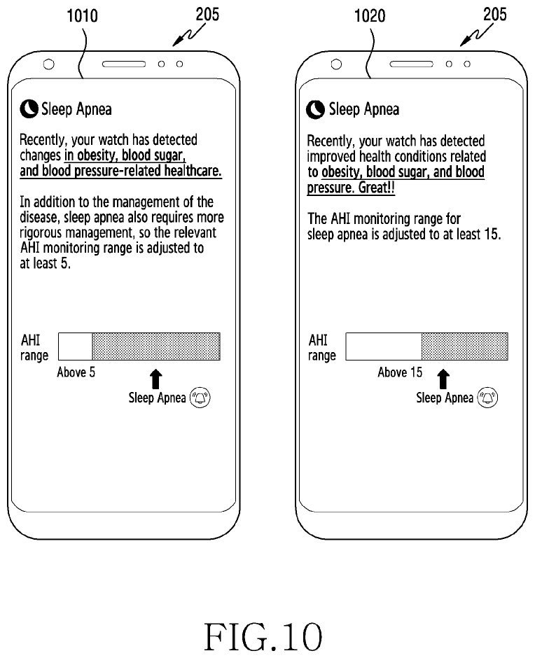

In operation 450, as the sleep breathing disorder event is detected, the electronic device 200 may provide a user interface (e.g., the first screen 710 and the second screen 720 in FIG. 7, the first screen 810 in FIG. 8, the first screen 910 and the second screen 920 in FIG. 9, or the first screen 1010 and the second screen 1020 in FIG. 10) for the sleep breathing disorder event.

The user interface may be intended to notify of a result of monitoring a sleep breathing disorder. For example, the user interface may include information about whether or not a sleep breathing disorder occurs and/or a risk of a sleep breathing disorder.

The user interface for a sleep breathing disorder may be provided through at least one of the output module 250 of the electronic device 200 and/or an external electronic device connected to the electronic device 200 through short-range wireless communication.

For example, the electronic device 200 (e.g., a wearable device such as a smart watch) may autonomously output a user interface for a sleep breathing disorder event through the output module 250. As another example, the electronic device 200 may transmit information about a user interface for a sleep breathing disorder event to an external electronic device (e.g., a wearable device worn on the user who is sleeping or a mobile device placed close to the user who is sleeping) through the communication circuit 220 (e.g., a short-range wireless communication module) such that the external electronic device may output the user interface. As another example, the electronic device 200 may provide a user interface for a sleep breathing disorder event simultaneously through the output module 250 and an external electronic device. The user interface for a sleep breathing disorder event may be intended for notification of a sleep breathing disorder event. The user interface may be implemented as a visual type (e.g., a screen or notification messages), an auditory type (e.g., audio signals such as music or sound), a tactile type (e.g., vibration), or a hybrid type combining at least some of them.

In an embodiment, the electronic device 200 may determine whether or not to deactivate the sleep breathing disorder notification function, based on temporary state information (e.g., alcohol intake, caffeine intake, stress level, life habit, location, and place), which may be environmental and/or physiological factors causing mis-determination of the sleep breathing disorder.

The electronic device 200 may deactivate the sleep breathing disorder notification function, based on the determination. The processor 210 may deactivate the sleep breathing disorder notification function, based on the determination. For example, if the sleep breathing disorder notification function is deactivated (or turned oft), the entire sleep monitoring function may be turned off, or only a notification function in the sleep monitoring function may be selectively turned off. As the sleep breathing disorder notification function is deactivated, the electronic device 200 may skip (or omit) at least one of the operation 430 of determining the reference condition, the operation 440 of detecting the sleep breathing disorder event, or the operation 450 of providing the user interface.

In an embodiment, the electronic device 200 may turn on/off a breathing disorder notification function according to temporary state information that may be environmental and/or physiological factors causing mis-determination about a sleep breathing disorder, thereby reducing a false alarm rate. For example, if the user consumes alcohol, it may involve a temporary sleep breathing disorder, so the sleep breathing disorder notification function may be turned off. As another example, if the sleeping place is the outdoors or the place other than a usual place, the sleep breathing disorder notification function may be turned off.

If false alarms occur frequently, users may not be aware of the risk caused by a sleep breathing disorder.

According to an embodiment, it is possible to accurately determine the sleep breathing disorder, thereby reducing the risk of mis-determination or the false alarm rate. It is possible to support effective correction and treatment by providing a user experience of a sleep breathing disorder at a right time.

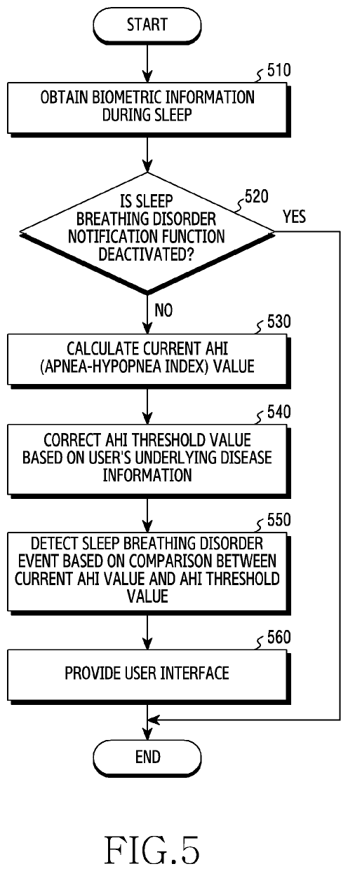

FIG. 5 is a flowchart illustrating an operation method of an electronic device according to an embodiment.

At least some of the operations in FIG. 5 may correspond to the operations in FIG. 4. For example, operation 510 in FIG. 5 may correspond to operation 420 in FIG. 4. Operation 540 in FIG. 5 may correspond to operation 430 in FIG. 4. Operation 550 in FIG. 5 may correspond to operation 440 in FIG. 4. Operation 560 in FIG. 5 may correspond to operation 450 in FIG. 4.

Referring to FIG. 5, in operation 510, the electronic device 200 may collect and process biometric information (e.g., oxygen saturation, heart rate, heart rate variability, blood pressure, and/or blood sugar) during the user's sleep via one or more sensors included on the electronic device 200.

Sleep is a biological activity for physiological recovery, and the parasympathetic nervous system may be activated during sleep. Accordingly, biometric information of a pattern different from that of daily activity may be detected during the sleep activity.

The operation of processing the biometric information may be performed immediately or at a specified time (e.g., at the end of sleep or at a wake-up time). For example, the electronic device 200 may collect and process biometric information in real time (or immediately) while the user is in a sleep state and store the same in the memory 240. As another example, the electronic device 200 may store raw data in the memory 240 to optimize current consumption and process the biometric information in a batching manner after sleep ends.

In operation 520, the electronic device 200 may determine whether or not to deactivate the sleep breathing disorder notification function.

In an embodiment, the electronic device 200 may determine whether or not to deactivate (turn off) the sleep breathing disorder notification function, based on the user's temporary state information (e.g., alcohol intake, caffeine intake, stress level, lifestyle, location, and/or place). The electronic device 200 may identify the user's temporary state information in various ways, and a method thereof is not limited. For example, alcohol/caffeine intake information may be input by the user onto the execution screen of an application of the electronic device 200. As another example, the electronic device 200 may identify the time of returning home, bedtime, payment history, visit places, and/or movement route of the user to estimate whether or not the temporary state of the day conforms to the daily state (or normal state). As another example, the electronic device 200 may recognize the current location or place of the user using a GNSS module.

The electronic device 200 may deactivate the sleep breathing disorder notification function, based on the determination. For example, if the user consumes alcohol, it may cause a temporary sleep breathing disorder and frequent false alarms according thereto, so the sleep breathing disorder notification function may be turned off in advance, thereby preventing such a phenomenon. As another example, if the sleeping place is the outdoors or the place other than a usual place, the sleep breathing disorder notification function may be turned off.

If the sleep breathing disorder notification function of the electronic device 200 is deactivated, the operation of monitoring a sleep breathing disorder may be terminated.

If the sleep breathing disorder notification function is activated, the electronic device 200 may proceed to operation 530 for monitoring a sleep breathing disorder.

In operation 530, the electronic device 200 may calculate an AHI value (or a current AHI value) from the sleep biometric information collected in operation 510. The AHI value may be an index indicating whether or not there is a sleep disorder called apnea (or hypopnea) and how severe it is.

For example, due to sleep apnea (or hypopnea), the oxygen saturation may be lower than a reference level for a certain period of time or longer, or the number of times the oxygen saturation level is lower than a reference level may exceed a specified number of times. The electronic device 200 may detect an apnea state in which breathing stops for a specified period of time (e.g., 10 seconds) or longer, or a hypopnea state in which the respiration volume abnormally decreases for a specified period of time (e.g., 10 seconds) or longer using the oxygen saturation continuously measured during sleep.

Referring to Equation 1 below, the AHI value may be a value obtained by dividing the number of times apnea (or hypopnea) occurs during one night by the sleep duration.

$$AHI = \frac{E}{T - W} \qquad [ Equation 1$$

Here, E is the total number of respiratory events (apnea-hypopnea), T is the total time of sleep, and W is the time during which the sleep awakening stage was maintained (or the time the user spent in the wake stage of sleep).

In operation 540, the electronic device 200 may correct a threshold value that is a reference for detecting a sleep breathing disorder event, based on the user's underlying disease information (e.g., hypertension, diabetes, and/or obesity).

The electronic device 200 may adaptively apply a threshold value for detecting a sleep breathing disorder depending on whether or not the user has an underlying disease and/or the underlying disease state. For example, the electronic device 200 may actively change the AHI threshold value from a preconfigured first value to a second value, based on whether or not the user has an underlying disease, and/or whether the user's health changes from not having an underlying disease to than having an underlying disease. As another example, the electronic device 200 may change the AHI threshold value from a preconfigured first value to a third value, based on a risk level according to the user's underlying disease state and/or a change in the risk level as the user's underlying disease state changes.

In operation 550, the electronic device 200 may compare the AHI value (or current AHI value) calculated in operation 530 with the AHI threshold value corrected in operation 540 and detect a sleep breathing disorder event, based on the comparison.

For example, the electronic device 200 may detect a sleep breathing disorder event based on occurrence of a unit event in which the AHI value (or current AHI value) exceeds the corrected threshold value. For example, if the unit event occurs, the electronic device 200 may immediately determine that a sleep breathing disorder event has occurred. As another example, the electronic device 200 may determine that a sleep breathing disorder event has occurred if the unit event occurs a specified number of times (e.g., 2 times) or more during a specified number of sleep cycles (e.g., 3 sleep cycles).

In an embodiment, the electronic device 200 may actively change or correct an AHI threshold value, which is a reference for monitoring a sleep breathing disorder, depending on whether or not the user has an underlying disease and/or the underlying disease state.

As an example, the electronic device 200 may identify the user as one of either an attention-required user who has an underlying disease (e.g., a hypertensive or diabetic patient and/or an obese user) or a general user who has no underlying disease, based on the presence or absence of the underlying disease. If the user is an attention-required user, the electronic device 200 may lower the AHI threshold value from a preconfigured first value to a second value. The electronic device 200 may detect a sleep breathing disorder event, based on the case where an AHI value (or current AHI value) calculated from biometric information exceeds the second value.

As another example, the electronic device 200 may actively adjust the AHI threshold value in stages depending on the risk level of the underlying disease. For example, a user who has no underlying disease may be classified as a first user with the lowest risk level, a user who has an underlying disease with a low severity may be classified as a second user with an intermediate risk level, and a user who has an underlying disease and a high severity may be classified as a third user with the highest risk level. For the first user, the AHI threshold value may be maintained to be a first value, and a sleep breathing disorder event may be detected based on the case where a current AHI value exceeds the first value. For the second user, the AHI threshold value may be lowered from the first value to a second value, and a sleep breathing disorder event may be detected based on the case where the current AHI value exceeds the second value. For the third user, the AHI threshold value may be lowered from the first value to a third value, which is lower than the second value, and a sleep breathing disorder event may be detected based on the case where the current AHI value exceeds the third value.

In an embodiment, the electronic device 200 may compare the current underlying disease state of the user with a previous underlying disease state of the user to identify whether or not there is a substantial change therein.

In order to increase the accuracy of monitoring a sleep breathing disorder, a comparison between the current underlying disease state of the user and a previous underlying disease state of the user may be required (or it may be required to identify whether or not the recent underlying disease state of the user is the same as a previous state) because various factors may affect sleep in daily life.

The user's underlying disease (or chronic disease) may be a chronic factor influencing a sleep breathing disorder. For example, if the user has hypertension or diabetes, the AHI threshold value may be adjusted as a personalized procedure for accurate determination. The underlying disease condition may be a concept that includes abnormal body conditions that affect a sleep breathing disorder such as obesity, overweight, and/or body mass index (BMI), as well as the condition of the underlying disease itself, such as hypertension or diabetes.

In an embodiment, if a change in the user's underlying disease state (e.g., blood pressure, blood sugar, obesity, and/or overweight) is identified, the electronic device 200 may change the AHI threshold value, based on the identification. The electronic device 200 may detect a sleep breathing disorder event or provide a user interface for notification of a sleep breathing disorder, based on the changed threshold value.

The reference condition for monitoring a sleep breathing disorder may be actively or adaptively changed depending on the user's underlying disease state (e.g., the presence or absence of an underlying disease, the type or degree thereof, or a change in abnormal physical state).

As an example, if a new underlying disease occurs to the user, the electronic device 200 may lower the AHI threshold value from 15 to 5. If the threshold value is lowered, the sensitivity of monitoring may increase, enabling more accurate monitoring. As another example, the electronic device 200 may increase the AHI threshold value from 5 to 15 if an underlying disease is improved (e.g., a decrease in blood sugar level in a diabetic patient or a decrease in blood pressure in a hypertensive patient). As another example, the electronic device 200 may lower the AHI threshold value from 15 to 10 if the user has hypertension and further lower the threshold value by 5 to change the threshold value into 5 if the user is obese. As another example, the electronic device 200 may lower the threshold value from 15 to 12 if the user is slightly overweight, lower the threshold value from 12 to 10 if the user is obese, or lower the threshold value to 5 if the user is highly obese.

In operation 560, the electronic device 200 may provide a user interface for a sleep breathing disorder event. The user interface may be intended for notification of a sleep breathing disorder event. For example, if the sleep breathing disorder notification function of the electronic device 200 is activated, a user interface for a sleep breathing disorder event may be provided. If the sleep breathing disorder notification function is deactivated, monitoring for a sleep breathing disorder event may not be performed, or a user interface may not be provided, regardless of whether or not a sleep breathing disorder event occurs.

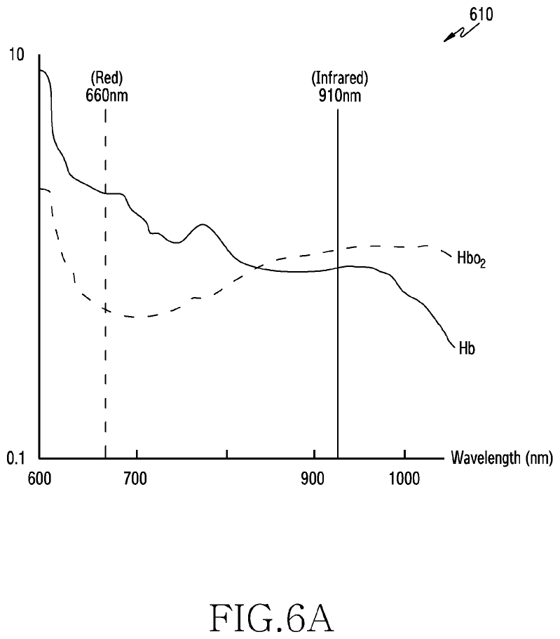

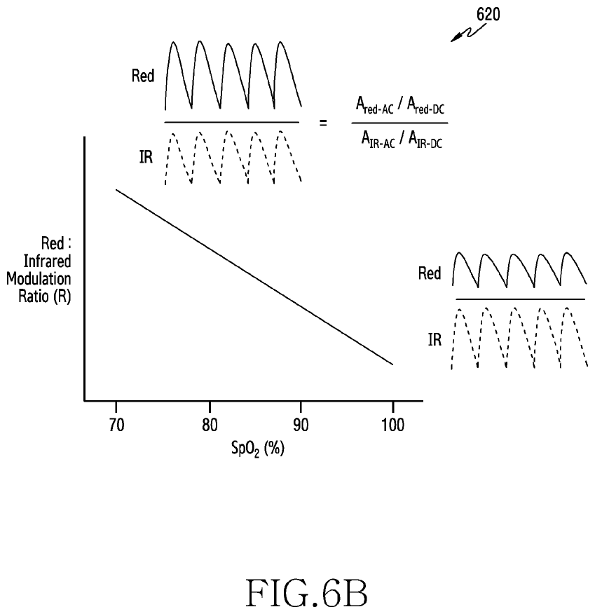

FIGS. 6A and 6B are diagrams illustrating a method of measuring oxygen saturation in an electronic device according to an embodiment.

Reference numeral 610 in FIG. 6A is a graph illustrating a difference in absorbance depending on wavelengths of red light and infrared light. Reference numeral 620 in FIG. 6B is a graph illustrating a relationship between blood oxygen saturation and a modulation rate (R-ratio). The modulation rate may indicate a ratio of an AC component to a DC component of an input signal of red light and infrared light.

In an embodiment, the electronic device 200 may irradiate a user's body with two types of light (e.g., red light and infrared light) of different wavelengths and measure the intensity of reflected or transmitted light using a biometric sensor (e.g., a PPG sensor). As shown in reference numeral 610, a difference in absorbance between two types of light may occur depending on the degree of oxygen binding of hemoglobin in the blood (or saturation that is a relative concentration of hemoglobin (Hb) and oxyhemoglobin (HbO2)). The electronic device 200 may measure the light intensity over time through a biometric sensor (e.g., a PPG sensor), obtain a modulation rate value from the measurement result, and calculate the user's blood oxygen saturation (SpO2) using the modulation rate value.

In an embodiment, the electronic device 200 may include two light-emitting diodes (LEDs) (e.g., a red LED and an infrared LED) in hardware in order to measure blood oxygen saturation.

In an embodiment, the electronic device 200 may continuously collect oxygen saturation information during sleep. For example, the electronic device 200 may calculate oxygen saturation for each sleep period while alternately irradiating with red light and infrared light through the red LED and infrared LED, and store information about a change in the oxygen saturation in the memory 240.

Red and infrared LEDs may be relatively susceptible to dynamic noise (motion). In an embodiment, the electronic device 200 may detect a motion through a motion sensor (e.g., an acceleration sensor and/or a gyro sensor) during the total sleep period and measure the oxygen saturation only if the motion is less than or equal to a specified reference, thereby improving reliability of a measurement result.

In an embodiment, the electronic device 200 may determine whether or not the user's sleep starts based on a user's heart rate variability (HRV) analysis result (e.g., an LF/HF value) using a biometric sensor (e.g., a PPG sensor) and/or the user's posture (e.g., a lying position and/or a posture in which the user's motion is less than or equal to a specified reference). If the user's sleep starts, the electronic device 200 may automatically enter an oxygen saturation measurement mode. The electronic device 200 may record output data of the biometric sensor with low power during sleep. The electronic device 200 may determine that the sleep ends through a motion sensor (e.g., an acceleration sensor and/or a gyro sensor), process output data of the biometric sensor if the sleep ends, and analyze the processed data to calculate oxygen saturation during sleep.

In an embodiment, the electronic device 200 may determine whether or not a sleep breathing disorder event occurs based on a change in the oxygen saturation during a sleep period (e.g., a sleep period of 4 hours after sleep starts or a total sleep period). For example, if the oxygen saturation is maintained to be lower than a reference for a certain period of time or if the number of times the oxygen saturation becomes lower than a reference exceeds a specified number of times, a current AHI value may be calculated using the oxygen saturation continuously measured during the sleep period, and a sleep breathing disorder may be monitored using the calculated AHI value.

FIGS. 7, 8, 9, and 10 described herein show examples of user interfaces that may be provided by the electronic device 200 (e.g., the wearable device 201 or the mobile device 205) according to various embodiments. For example, one or more of the screens shown in FIGS. 7, 8, 9, and 10 may be displayed in the electronic device 200 according to various embodiments. For example, the screens may be execution screens of applications (e.g., healthcare applications or sleep care applications) running in the electronic device 200 (e.g., one of the wearable device 201 or the mobile device 205) or applications (e.g., healthcare applications or sleep care applications) running in an external electronic device (e.g., the other of the wearable device 201 or the mobile device 205) connected to the electronic device 200 through short-range wireless communication.

FIG. 7 is an example of a user interface related to a sleep breathing disorder notification function of an electronic device according to an embodiment.

In an embodiment, the wearable device 201 may execute a sleep monitoring function (or a sleep breathing disorder notification function) and output a user interface such as a first screen 710, based on a result of the sleep monitoring. The first screen 710 may be a screen showing the result of the sleep monitoring by the wearable device 201.

For example, in order to lower mis-determination or a false alarm rate, the wearable device 201 may monitor breathing in a sleep state for several days (multi-nights) and determine whether or not a sleep breathing disorder event occurs. As a result of sleep monitoring for several days, if a unit event in which an AHI value exceeds a threshold value occurs a certain number of times (e.g., 2 times for 2 sleep cycles, or 2 times or more for 3 sleep cycles) or more, the wearable device 201 may determine that a sleep breathing disorder event has occurred and display the first screen 710 informing the user of the sleep breathing disorder state.

If a sleep breathing disorder event is detected as a result of sleep monitoring, the wearable device 201 may display the first screen 710.

As shown, the first screen 710 may include an indicator 711 (e.g., an icon, a live icon, or an object) notifying of the occurrence of a sleep breathing disorder event. The first screen 710 may include a first area 713 for notifying that the user is in a sleep breathing disorder state and a second area 715 for guiding a communication connection with a doctor or transmitting and providing sleep monitoring result information to an attending doctor.

The wearable device 201 and the mobile device 205 may be in a connected state through short-range wireless communication. The wearable device 201 and the mobile device 205 may be in the state in which the same user is logging on.

The mobile device 205 may execute a sleep monitoring function by itself or receive sleep monitoring result information by interworking with the wearable device 201.

The mobile device 205 may output a user interface such as a second screen 720.

As shown, the second screen 720 may include an indicator 721 for a sleep breathing disorder notification function (e.g., an icon, a live icon, or an object), a first area 723 informing the user of the recent occurrence of a sleep breathing disorder and recommending treatment, and a second area 725 showing a detailed history of the occurrence of a sleep breathing disorder for the user.

FIG. 8 is another example of a user interface related to a sleep breathing disorder notification function of an electronic device according to an embodiment.

For example, the mobile device 205 may output a user interface such as a first screen 810.

The first screen 810 may include information about a sleep monitoring result for several days (multi-nights) (e.g., 10 days or 10 sleep cycles) in order to lower mis-determination or the false alarm rate (e.g., the date and time at which a unit event in which an AHI value exceeds a threshold value occurs, and the number of occurrences).

FIG. 9 is another example of a user interface related to a sleep breathing disorder notification function of an electronic device according to an embodiment.

For example, the mobile device 205 may output a user interface such as a first screen 910 or a second screen 920.

The first screen 910 may be a screen informing that the wearable device 201 of the user was not worn last night.

The second screen 920 may be a screen recommending to wear the wearable device 201 for sleep monitoring today. The second screen 920 may include an indicator 921 (e.g., a menu or an object) for switching a sleep monitoring function (or a sleep breathing disorder notification function) from an off state to an on state.

The first screen 910 may be a screen providing guidance for a correct wearing method of the wearable device 201 (e.g., to wear close to the upper wrist bone of the arm for measurement of the oxygen saturation (SpO2)) or a necessary condition for detecting a sleep breathing disorder event (e.g., it is required to measure the oxygen saturation (SpO2) for 4 hours of sleep or longer and for a time corresponding to 70% of total sleep duration or longer).

For example, if the number of measurements for risk assessment is insufficient even though the user performed measurement more than once,

the wearable device 201 may provide a reminder to the user in consideration of the user's average bedtime to induce correct measurement.

If a condition required to measure an AHI value is not met (e.g., in the case of sleep for less than 4 hours or in the case where oxygen saturation is not measured during 70% of the entire sleep or more) even though the user performed measurement more than once, the wearable device 201 may provide precautions for the next sleep cycle and accurate guidance for wearing the wearable device 201 (e.g., to wear close to the upper wrist bone of the arm), thereby preventing repetition of measurement failures.

FIG. 10 is another example of a user interface related to a sleep breathing disorder notification function of an electronic device according to an embodiment.

For example, the mobile device 205 may output a user interface such as a first screen 1010 or a second screen 1020.

The first screen 1010 may be a screen providing guidance of an AHI range (e.g., 5 or less) for monitoring a sleep breathing disorder.

If there is a change in the user's underlying disease state, the second screen 1020 may be output, instead of the first screen 1010.

The second screen 1020 may be a screen informing that the AHI range for monitoring a sleep breathing disorder has been changed (e.g., adjusted from 5 or less to 15 or less) depending on a change in the user's underlying disease state.

For example, if the user has a new underlying disease, a threshold value may be adjusted from 15 to 5 in order to increase the sensitivity of monitoring a sleep breathing disorder. In this case, a notification of occurrence of a sleep breathing disorder may be provided based on the case where the AHI value exceeds the threshold value of 5.

As another example, if the underlying disease is improved through lifestyle changes, the threshold value may be adjusted from 5 to 15. In this case, a notification of occurrence of a sleep breathing disorder may be provided based on the case where the AHI value exceeds the threshold value of 15.

The user interfaces described with reference to in FIGS. 7, 8, 9, and 10 above are merely examples for description, and the scope of the embodiments is not limited thereto, and the user interface may be applied, modified, and/or expanded in various ways.

In an embodiment, the electronic device 200 (e.g., the wearable device 201 and/or the mobile device 205) may output a user interface (e.g., a screen) for guiding lifestyle improvement. For example, even if the AHI value does not exceed a threshold value (e.g., 15 or higher), the user who belongs to a risk group (e.g., the user whose AHI value is in the range of 5 to 14) may be provided with information that helps improve a sleep breathing disorder (e.g., breathing or meditation to relieve stress, caffeinated beverages, record food intake, recommend to reduce intake, and refrain from alcohol) in relation to factors (e.g., stress, caffeine, and alcohol) that may affect the sleep breathing disorder.

An electronic device (e.g., the electronic device 200 in FIG. 2) according to various embodiments may include a communication circuit (e.g., the communication circuit 220 in FIG. 2), at least one sensor (e.g., the sensor module 230 in FIG. 2), and at least one processor (e.g., the processor 210 in FIG. 2) connected to the communication circuit and the at least one sensor. The at least one processor may be configured to obtain biometric information of a user through the communication circuit or the at least one sensor during the user's sleep, determine a reference condition for detecting a sleep breathing disorder event, based on underlying disease information of the user, detect a sleep breathing disorder event, based on the biometric information and the reference condition, and provide a user interface for the sleep breathing disorder event as the sleep breathing disorder event is detected.

According to various embodiments, the at least one processor may be configured to identify whether or not the user has an underlying disease, based on the underlying disease information, and adjust the reference condition from a preconfigured first condition to a second condition, based on whether or not the user has an underlying disease.

According to various embodiments, if the user has an underlying disease, an apnea-hypopnea index (AHI) threshold value included in the reference condition may be lowered from a first value to a second value. The sleep breathing disorder event may be detected based on the case where an AHI value obtained from the biometric information exceeds the second value.

According to various embodiments, the at least one processor may be configured to determine a risk level of a sleep breathing disorder, based on the underlying disease information, and adjust the reference condition from a preconfigured first condition to a second condition, based on the risk level.

According to various embodiments, an AHI threshold value included in the reference condition may be lowered from a first value to a third value, based on the risk level. The sleep breathing disorder event may be detected based on the case where the AHI value obtained from the biometric information exceeds the third value.

According to various embodiments, the reference condition may be adjusted as the underlying disease information is updated. The sleep breathing disorder event may be detected based on the adjusted reference condition.

According to various embodiments, it may be determined whether or not to deactivate a sleep breathing disorder notification function, based on temporary state information of the user. The sleep breathing disorder notification function may be deactivated based on the determination.

According to various embodiments, at least one of determining the reference condition, detecting the sleep breathing disorder event, or providing the user interface may be skipped as the sleep breathing disorder notification function is deactivated.

According to various embodiments, the temporary state information may include information about at least one of alcohol intake, caffeine intake, stress level, lifestyle, location, and place.

According to various embodiments, the at least one processor may detect the sleep breathing disorder event by further considering a user's previous sleep record. The previous sleep record may include at least one piece of first sleep data related to a user's past sleep activity repeated a specified number of times or more, second sleep data related to a user's past sleep activity during a recent predetermined period, and sleep breathing disorder analysis result data based on the first sleep data or the second sleep data.

An operation method of an electronic device according to various embodiments may include obtaining biometric information of a user during the user's sleep, determining a reference condition for detecting a sleep breathing disorder event, based on underlying disease information of the user, detecting a sleep breathing disorder event, based on the biometric information and the reference condition, and providing a user interface for the sleep breathing disorder event as the sleep breathing disorder event is detected.

According to various embodiments, the determining of the reference condition may include identifying whether or not the user has an underlying disease, based on the underlying disease information, and adjusting the reference condition from a preconfigured first condition to a second condition, based on whether or not the user has an underlying disease.

According to various embodiments, if the user has an underlying disease, an apnea-hypopnea index (AHI) threshold value included in the reference condition may be lowered from a first value to a second value. The sleep breathing disorder event may be detected based on the case where an AHI value obtained from the biometric information exceeds the second value.

According to various embodiments, the determining of the reference condition may include determining a risk level of a sleep breathing disorder, based on the underlying disease information, and adjusting the reference condition from a preconfigured first condition to a second condition, based on the risk level.

According to various embodiments, an AHI threshold value included in the reference condition may be lowered from a first value to a third value, based on the risk level. The sleep breathing disorder event may be detected based on the case where an AHI value obtained from the biometric information exceeds the third value.

According to various embodiments, the method may further include adjusting the reference condition as the underlying disease information is updated. The sleep breathing disorder event may be detected based on the adjusted reference condition.

According to various embodiments, it may be determined whether or not to deactivate a sleep breathing disorder notification function, based on temporary state information of the user. The sleep breathing disorder notification function may be deactivated based on the determination.

According to various embodiments, as the sleep breathing disorder notification function is deactivated, at least one of determining the reference condition, detecting the sleep breathing disorder event, or providing the user interface may be skipped.

According to various embodiments, the temporary state information may include information about at least one of alcohol intake, caffeine intake, stress level, lifestyle, location, and place.

According to various embodiments, the sleep breathing disorder event may be detected by further considering a user's previous sleep record in the detecting. The previous sleep record may include at least one piece of first sleep data related to a user's past sleep activity repeated a specified number of times or more, second sleep data related to a user's past sleep activity during a recent predetermined period, and sleep breathing disorder analysis result data based on the first sleep data or the second sleep data.

1. An electronic device comprising:
a communication circuit;
at least one sensor; and
at least one processor connected to the communication circuit and the at least one sensor,
wherein the at least one processor is configured to:
obtain biometric information of a user through the communication circuit or the at least one sensor during a user's sleep;
determine a reference condition for detecting a sleep breathing disorder event, based on underlying disease information of the user;
detect the sleep breathing disorder event, based on the biometric information and the reference condition; and
provide a user interface for the sleep breathing disorder event as the sleep breathing disorder event is detected.

2. The electronic device of claim 1, wherein the at least one processor is configured to:
identify whether or not the user has an underlying disease, based on the underlying disease information; and
adjust the reference condition from a preconfigured first condition to a second condition, based on whether or not the user has the underlying disease.

3. The electronic device of claim 2, wherein in a case that the user has the underlying disease, an apnea-hypopnea index (AHI) threshold value included in the reference condition is lowered from a first value to a second value, and
the sleep breathing disorder event is detected based on the case where an AHI value obtained from the biometric information exceeds the second value.

4. The electronic device of claim 1, wherein the at least one processor is configured to:
determine a risk level of a sleep breathing disorder, based on the underlying disease information; and
adjust the reference condition from a preconfigured first condition to a second condition, based on the risk level.

5. The electronic device of claim 4, wherein the at least one processor is configured to:
lower an AHI threshold value included in the reference condition from a first value to a third value, based on the risk level; and
detect the sleep breathing disorder event, based on the case where the AHI value obtained from the biometric information exceeds the third value.

6. The electronic device of claim 1, wherein the at least one processor is configured to:
adjust the reference condition as the underlying disease information is updated; and
detect the sleep breathing disorder event, based on the adjusted reference condition.

7. The electronic device of claim 1, wherein the at least one processor is configured to:
determine whether or not to deactivate a sleep breathing disorder notification function, based on temporary state information of the user; and
deactivate the sleep breathing disorder notification function, based on the determination whether or not to deactivate a sleep breathing disorder notification function.

8. The electronic device of claim 7, wherein the at least one processor is configured to skip at least one of determining the reference condition, detecting the sleep breathing disorder event, or providing the user interface as the sleep breathing disorder notification function is deactivated.

9. The electronic device of claim 7, wherein the temporary state information comprises information about at least one of alcohol intake, caffeine intake, stress level, lifestyle, location, and place.

10. The electronic device of claim 1, wherein the at least one processor is configured to detect the sleep breathing disorder event by further considering a user's previous sleep record, and
wherein the previous sleep record comprises at least one piece of:
first sleep data related to a user's past sleep activity repeated a specified number of times or more;
second sleep data related to the user's past sleep activity during a recent predetermined period; and
sleep breathing disorder analysis result data based on the first sleep data or the second sleep data.

11. An operation method of an electronic device, the method comprising:
obtaining biometric information of a user during a sleep period of the user;
determining a reference condition for detecting a sleep breathing disorder event, based on underlying disease information of the user;
detecting the sleep breathing disorder event, based on the biometric information and the reference condition; and
providing a user interface for the sleep breathing disorder event as the sleep breathing disorder event is detected.

12. The method of claim 11, wherein the determining of the reference condition comprises:
identifying whether or not the user has an underlying disease, based on the underlying disease information; and
adjusting the reference condition from a preconfigured first condition to a second condition, based on whether or not the user has the underlying disease.

13. The method of claim 12, comprising:
in a case that the user has the underlying disease, lowering an apnea-hypopnea index (AHI) threshold value included in the reference condition from a first value to a second value; and
detecting the sleep breathing disorder event, based on the case where an AHI value obtained from the biometric information exceeds the second value.

14. The method of claim 11, wherein the determining of the reference condition comprises:
determining a risk level of a sleep breathing disorder, based on the underlying disease information; and
adjusting the reference condition from a preconfigured first condition to a second condition, based on the risk level.

15. The method of claim 14, comprising:
lowering an AHI threshold value included in the reference condition from a first value to a third value, based on the risk level; and
detecting the sleep breathing disorder event, based on the case where an AHI value obtained from the biometric information exceeds the third value.

16. The method of claim 11, comprising:
adjusting the reference condition as the underlying disease information is updated, and
detecting the sleep breathing disorder event, based on the adjusted reference condition.

17. The method of claim 11, comprising:
determining whether or not to deactivate a sleep breathing disorder notification function, based on temporary state information of the user; and
deactivating the sleep breathing disorder notification function, based on the determination of whether or not to deactivate a sleep breathing disorder notification function.

18. The method of claim 17, wherein as the sleep breathing disorder notification function is deactivated, at least one of determining the reference condition, detecting the sleep breathing disorder event, or providing the user interface is skipped.

19. The method of claim 17, wherein the temporary state information comprises information about at least one of alcohol intake, caffeine intake, stress level, lifestyle, location, and place.

20. The method of claim 11, wherein the sleep breathing disorder event is detected by further considering a user's previous sleep record in the detecting, and
wherein the previous sleep record comprises at least one piece of:
first sleep data related to a user's past sleep activity repeated a specified number of times or more;
second sleep data related to a user's past sleep activity during a recent predetermined period; and
sleep breathing disorder analysis result data based on the first sleep data or the second sleep data.

21. An electronic device comprising:
a communication circuit;
at least one sensor; and
at least one processor connected to the communication circuit and the at least one sensor, the processor configured to:
determine an underlying disease state of a user;
determine a reference condition based at least in part on the underlying disease state; and
adaptively change the reference condition in response to detecting a change in the underlying disease state.

22. The electronic device of claim 21, wherein the reference condition is an apnea-hypopnea index (AHI) threshold value.

23. The electronic device of claim 22, wherein the at least one sensor is configured to monitor biometric information of the user during a period of sleep, and wherein the processor is configured to assign an AHI value to the biometric information and to detect a sleep breathing disorder event in response to the AHI value exceeding the changed AHI threshold value.

24. The electronic device of claim 23, wherein determining the underlying disease state includes identifying the user as one of an attention-required having symptoms of having a sleep breathing disorder or a general user excluding the symptoms of a sleep breathing disorder.

25. The electronic device of claim 24, wherein the underlying disease state includes one or a combination of a hypertensive patient, a user experiencing hypertension, a user experiencing hyperglycemia, a diabetic patient, an overweight user, a user whose stress level exceeds a specified threshold value, and a user whose average sleep duration is less than or equal to a threshold value may be the attention-required users.
